(19) United States
(12) Patent Application Publication
(10) Pub. No.: US 2024/0071590 A1
(43) Pub. Date: Feb. 29, 2024

(54) MEDICINE INTAKE SUPPORT DEVICE

(71) Applicant: CANON MEDICAL SYSTEMS CORPORATION, Otawara-shi (JP)

(72) Inventors: Mariko SHIBATA, Nasushiobara (JP); Kohei WATANABE, Utsunomiya (JP)

(73) Assignee: CANON MEDICAL SYSTEMS CORPORATION, Otawara-shi (JP)

(21) Appl. No.: 18/458,184

(22) Filed: Aug. 30, 2023

(30) Foreign Application Priority Data
Aug. 30, 2022 (JP) 2022-137289

(51) Int. Cl. G16H 20/10; A61J 7/04; G16H 40/67; G16H 50/20; G16H 70/40

(52) U.S. Cl. CPC G16H 20/10; A61J 7/0436; A61J 7/0481; G16H 40/67; G16H 50/20; G16H 70/40; A61J 2200/30

(57) ABSTRACT

The medicine intake support device includes a processing circuitry and a memory. The processing circuitry acquires a previous medicine intake time and a next scheduled medicine intake time, wherein the previous medicine intake time is a time when a patient previously took a prescription medicine, and the next scheduled medicine intake time is a predetermined time when the patient will take a next dose of the prescription medicine. The memory stores a type of the prescription medicine and a measure in response to the patient's failure to take the type of the prescription medicine in correspondence with each other. If the patient failed to take the prescription medicine, the processing circuitry suggests a response method in response to the patient's failure to take the prescription medicine based on the measure stored in the memory, the previous medicine intake time, the next scheduled medicine intake time, and a current time.

### CROSS REFERENCE TO RELATED APPLICATIONS

This application is based upon and claims the benefit of priority from the prior Japanese Patent Application No. 2022-137289, filed on Aug. 30, 2022, the entire contents of which are incorporated herein by reference.

### FIELD

The embodiments disclosed in the present specification and drawings relate to a medicine intake support device.

### BACKGROUND

Antihypertensive agents prescribed for patients with high blood pressure, insulin preparations prescribed for diabetic patients, and medicine such as anticancer agents require strict adherence to a medicine intake time. Missing the medicine intake time and compromising the medicine intake time for the next dose is undesirable for ensuring proper therapeutic efficacy.

However, although technologies to prevent failure of medicine intake were proposed conventionally, there was no effective solutions to support patients who forgot to take the medicine with effective response methods.

### BRIEF DESCRIPTION OF THE DRAWINGS

FIG. 1 is a block diagram illustrating an exemplary configuration of a medicine intake support device according to a first embodiment.

FIG. 2 is a diagram illustrating an exemplary database stored by a memory circuitry in the medicine intake support device according to the first embodiment.

FIG. 3 is a flowchart illustrating an exemplary operation of the medicine intake support device according to the first embodiment.

FIG. 4 is a flowchart continued from that of FIG. 3, illustrating the exemplary operation of the medicine intake support device according to the first embodiment.

FIG. 5 is a flowchart continued from that of FIG. 3, illustrating the exemplary operation of the medicine intake support device according to the first embodiment.

FIG. 6 is a description diagram for describing a registration process of a patient information in the exemplary operation of the medicine intake support device according to the first embodiment.

FIG. 7 is a description diagram for describing the registration process of a prescription medicine information in the exemplary operation of the medicine intake support device according to the first embodiment.

FIG. 8 is a description diagram different from FIG. 7, for describing the registration process of the prescription medicine information in the exemplary operation of the medicine intake support device according to the first embodiment.

FIG. 9 is a description diagram for describing the registration process of a medicine intake schedule information in the exemplary operation of the medicine intake support device according to the first embodiment.

FIG. 10 is a description diagram for describing a calculating process of a next recommended medicine intake time in the exemplary operation of the medicine intake support device according to the first embodiment.

FIG. 11 is a description diagram for describing a suggestion process of a response method against medicine intake failure in the exemplary operation of the medicine intake support device according to the first embodiment.

FIG. 12 is a description diagram for describing the suggestion process of a response method against excessive medicine intake in the exemplary operation of the medicine intake support device according to the first embodiment.

FIG. 13 is a block diagram illustrating an exemplary configuration of the medicine intake support device according to a second embodiment.

FIG. 14 is a flowchart illustrating the exemplary operation of the medicine intake support device according to the second embodiment.

FIG. 15 is a flowchart illustrating the exemplary operation of the medicine intake support device according to a third embodiment.

### DETAILED DESCRIPTION

With reference to the drawings below, embodiments of a medicine intake support device will be described. In the description below, note that same reference signs are given for components substantially identical in terms of configuration and function and duplicate description will be given only when necessary.

### First Embodiment

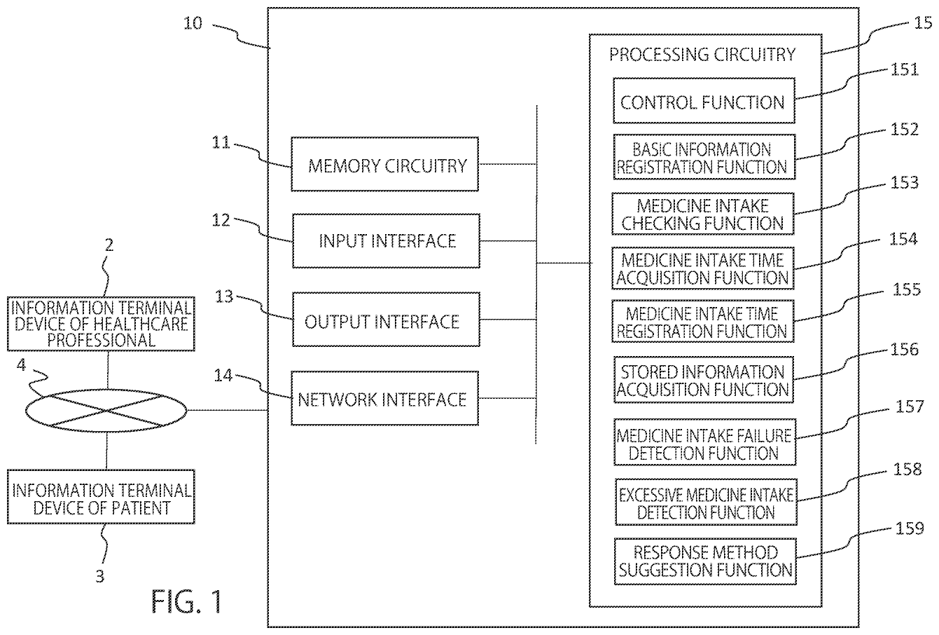

FIG. 1 is a block diagram illustrating an exemplary configuration of a medicine intake support device 10 according to a first embodiment. The medicine intake support device 10 is a device that supports intake of prescription medicines (i.e., controlled drugs) for patients. Specifically, the medicine intake support device 10 is a device that allows supporting adequate intake of medicine after the patient's intake status of the prescription medicine deviates from a predetermined intake status by suggesting response methods against the patient's deviation from the predetermined intake status to a healthcare professional or the like.

As shown in FIG. 1, the medicine intake support device 10 includes a memory circuitry 11, an input interface 12, an output interface 13, a network interface 14, and a processing circuitry 15. The memory circuitry is an example of a memory. The medicine intake support device 10 may be connected to an information terminal device 2 of the healthcare professional and an information terminal device 3 of the patient via a network 4. The information terminal devices 2, 3 may be portable terminal devices such as a mobile phone, a tablet terminal, or a wearable terminal. The information terminal device 3 of the patient may be an information terminal device owned by the patient or an information terminal device owned by someone other than the patient, such as a caregiver of the patient.

The memory circuitry 11 is a non-transitory memory device that stores various information and may be a Hard Disk Drive (HDD), a Solid-State Drive (SSD), an integrated circuit memory device, or the like. The memory circuitry 11 may store a control program that controls the medicine intake support device 10 and various data used to execute the control program. The memory circuitry 11 also may be a drive device other than the HDD and SSD that reads and writes various information between portable storage media such as a Compact Disc (CD), a Digital Versatile Disk (DVD), and a flash memory, or semiconductor memory elements such as a Random Access Memory (RAM).

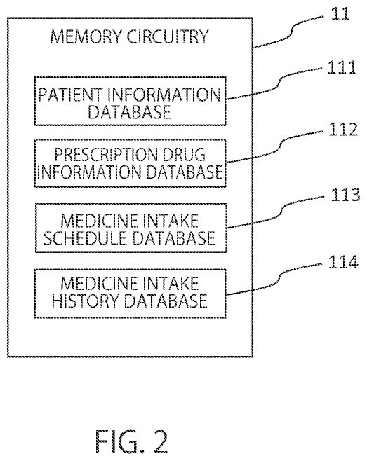

FIG. 2 is a diagram illustrating an exemplary database stored by a memory circuitry 11 in the medicine intake support device 10 according to the first embodiment. As shown in FIG. 2, the memory circuitry 11 includes a patient information database 111, a prescription medicine information database 112, a medicine intake schedule database 113, and a medicine intake history database 114, as exemplary data used to execute the control program that controls the medicine intake support device 10.

The patient information database 111 stores patient information representing unique characteristics of the patient. The patient information may include an age of the patient, a constitution of the patient (e.g., allergens and DNA info), a medical history of the patient, a disease of the patient, a comorbid disease of the patient, a metabolic function information of the patient (e.g., liver function and renal function), a comorbid drug of the patient, or a medicine intake guidance information from a doctor. The medicine intake guidance information may be information such as a number of dosages per day and a dosage amount of the prescription medicine. When storing the patient information of a plurality of patients in the patient information database 111, the patient information of each patient may be stored while associating different identification information for each patient.

The prescription medicine information database 112 stores prescription medicine information, which is information about the prescription medicine. The prescription medicine information includes a type of the prescription medicine (e.g., antihypertensive agent), properties of the prescription medicine (i.e., properties of the controlled drug), a time to wait before a next medicine intake, and a measure in response to the failure to take the prescription medicine, the measure corresponding to the type of the prescription medicine (hereinafter, also referred to as a measure against medicine intake failure). In other words, the prescription medicine information database 112 stores the type of the prescription medicine and the measure against medicine intake failure in correspondence with each other. Properties of the prescription medicine may include a severity of side effects of the prescription medicine, a likelihood of the side effects of the prescription medicine, an effective blood concentration of the prescription medicine, and an effective tissue concentration of the prescription medicine. The likelihood of side effects of the prescription medicine may include a blood concentration time curve showing a change of blood concentration of the prescription medicine over time, a blood concentration of the prescription medicine possibly causing the side effects, and a tissue concentration of the prescription medicine possibly causing the side effects. Properties of the prescription medicine may contain different contents corresponding to characteristics of the patient. For instance, consider storing the blood concentration and the tissue concentration of the prescription medicine as unique values calculated in advance corresponding to the patient's own metabolic function. Unique values for each patient may be continued to be recalculated by adequate inspections. Also, the blood concentration and the tissue concentration may be calculated by summing a drug components if the patient takes a plurality of prescription medicines. If the prescription medicine information of the plurality of patients is stored in the prescription medicine information database 112, the prescription medicine information of each patient may be stored while associating different identification information for each patient.

The measure against medicine intake failure stored in the prescription medicine information database 112 may be information such as “take the medicine immediately after noticing medicine intake failure. However, if a next scheduled medicine intake time is close, take the medicine at the next scheduled medicine intake time without taking it now.” Further, the measure against medicine intake failure stored in the prescription medicine information database 112 is a common (i.e., basic) response method corresponding to the type of the prescription medicine and does not consider specific times corresponding to a specific intake status of the patient. In other words, the measure against medicine intake failure stored in the prescription medicine information database 112 is different from a response method against medicine intake failure that considers a past medicine intake time, a next scheduled medicine intake time, and a current time, which is suggested by a response method suggestion function 159 as will be described later.

The prescription medicine information database 112 may further stores a measure in response to excessive intake of the prescription medicine, the measure corresponding to the type of the prescription medicine (hereinafter, also referred to as a measure against excessive medicine intake). The measure against excessive medicine intake stored in the prescription medicine information database 112 may be information such as “do not take the medicine at the next scheduled medicine intake time.” Note that the measure against excessive medicine intake stored in the prescription medicine information database 112 is a common (i.e., basic) response method corresponding to the type of the prescription medicine and does not consider specific times corresponding to a specific intake status of the patient. In other words, the measure against excessive medicine intake stored in the prescription medicine information database 112 is different from a response method against excessive medicine intake that considers the past medicine intake time, the next scheduled medicine intake time, and the current time, which is suggested by the response method suggestion function 159 as will be described later.

The medicine intake schedule database 113 stores medicine intake schedule information showing schedules for the patient to take the prescription medicine. The medicine intake schedule information includes scheduled medicine intake times, which are predetermined times for the patient to take the prescription medicine. There may be a plurality of scheduled medicine intake times per day. The scheduled medicine intake time may be once a day or once a plurality of days. If a plurality of prescription medicines is prescribed for the patient, the scheduled medicine intake information may include scheduled medicine intake information for each prescription medicine. If the scheduled medicine intake information for the plurality of patients is stored in the medicine intake schedule database 113, the scheduled medicine intake information for each patient may be stored while associating different identification information for each patient.

The medicine intake history database 114 stores medicine intake history information showing the patient's medicine intake history of the prescription medicine. The medicine intake history information includes medicine intake times, which are times when the patient takes the prescription medicine. If the medicine intake history information of the plurality of patients is stored in the medicine intake history database 114, the medicine intake history information of each patient may be stored while associating different identification information for each patient.

The input interface 12 is a device that receives various input operations from an operator. The input interface 12 converts the received input operations into electrical signals and outputs to the processing circuitry 15. The input interface 12 may receive input operations such as the patient information, the prescription medicine information, and the medicine intake schedule information. A mouse, a keyboard, a trackball, a switch, a button, a joystick, a touchpad, or a touch panel display, etc. may be appropriately used as the input interface 12. The input interface 12 may also include input devices used to detect the patient taking the prescription medicine. The input devices used to detect the intake of medicine may be, for example, a medicine intake storage system that detects and notifies that the prescription medicine has been removed from a storage container with sensors provided in the storage container, a detection system that detects intake behavior using a surveillance camera, or a notification system that detects and notifies that the prescription medicine was taken with a sensor attached to the prescription medicine itself. The input interface 12 may also be configured by an audio input device such as a microphone.

The output interface 13 is a device that outputs various information. The output interface 13 may display a Graphical User Interface (GUI) to receive various operations from the operator and images generated by the processing circuitry 15. The images generated by the processing circuitry 15 may include images suggesting response methods against the patient's intake status of the prescription medicine deviating from a predetermined intake status. A Liquid Crystal Display (LCD), a Cathode Ray Tube (CRT) display, an Organic Electro Luminescence Display (OLED), a Plasma display, or other arbitrary displays may be adequately used as the output interface 13. The display may be in a desktop form or may be configured as a tablet terminal etc. allowed to wirelessly communicate with the processing circuitry 15. The output interface 13 may also be configured by an audio output device such as a speaker.

The network interface 14 controls communication between the information terminal device 2 of the healthcare professional and the information terminal device 3 of the patient via the network 4 based on a command from the processing circuitry 15. The network interface 14 may send images suggesting response methods against the patient's intake status of the prescription medicine deviating from the predetermined intake status to at least one of the information terminal device 2 of the healthcare professional and the information terminal device 3 of the patient 3. The network interface 14 may be realized by, for instance, a network card, a network adapter, a Network Interface Controller (NIC), or the like.

The processing circuitry 15 is a circuit that controls the operation of the medicine intake support device 10. The processing circuitry 15 may include a control function 151, a basic information registration function 152, a medicine intake checking function 153, a medicine intake time acquisition function 154, a medicine intake time registration function 155, a stored information acquisition function 156, a medicine intake failure detection function 157, an excessive medicine intake detection function 158, and a response method suggestion function 159.

Here, each processing function executed by the control function 151, the basic information registration function 152, the medicine intake checking function 153, the medicine intake time acquisition function 154, the medicine intake time registration function 155, the stored information acquisition function 156, the medicine intake failure detection function 157, an excessive medicine intake detection function 158, and the response method suggestion function 159 of the processing circuitry 15 shown in FIG. 1 is stored in the memory circuitry 11 in a form of computer executable program. The processing circuitry 15 may be a processor. The processor configuring the processing circuitry 15 realizes functions corresponding to each program that is read by reading and executing each program from the memory circuitry 11. In other words, the memory circuitry 11 that has read each program includes each function shown in the processing circuitry 15 of FIG. 1.

Although each processing function of the control function 151, the basic information registration function 152, the medicine intake checking function 153, the medicine intake time acquisition function 154, the medicine intake time registration function 155, the stored information acquisition function 156, the medicine intake failure detection function 157, an excessive medicine intake detection function 158, and the response method suggestion function 159 were shown in FIG. 1 to be realized by the single processing circuitry 15, embodiments are not limited to this. For instance, the processing circuitry 15 may realize each processing function by combining a plurality of independent processors and letting each processor execute each program. Also, each processing function of the processing circuitry 15 may be realized by adequately distributing or integrating the single circuitry or the plurality of processing circuitry.

The control function 151 controls the operation of the basic information registration function 152, the medicine intake checking function 153, the medicine intake time acquisition function 154, the medicine intake time registration function 155, the stored information acquisition function 156, the medicine intake failure detection function 157, an excessive medicine intake detection function 158, and the response method suggestion function 159 by the program realizing the control function 151 read from the memory circuitry 11.

The basic information registration function 152 acquires the patient information, the prescription medicine information, and the medicine intake schedule information described above from the input interface 12 under the control of the control function 151. The basic information registration function 152 registers the acquired patient information to the patient information database 111 of the memory circuitry 11. The basic information registration function 152 also registers the acquired prescription medicine information to the prescription medicine information database 112 of the memory circuitry 11. The basic information registration function 152 also registers the acquired medicine intake schedule information to the medicine intake schedule database 113 of the memory circuitry 11.

The medicine intake checking function 153 detects that the patient took the prescription medicine under the control of the control function 151.

The medicine intake time acquisition function 154 acquires a time of detecting that the patient took the prescription medicine as a medicine intake time, by the medicine intake checking function 153 under the control of the control function 151.

The medicine intake time registration function 155 registers the medicine intake time acquired by the medicine intake time acquisition function 154 to the medicine intake history database 114 of the memory circuitry 11 under the control of the control function 151.

The stored information acquisition function 156 acquires information used for each of the medicine intake failure detection function 157, the excessive medicine intake detection function 158, and the response method suggestion function 159 under the control of the control function 151 by extracting (i.e., selecting) from the memory circuitry 11.

The medicine intake failure detection function 157 detects under the control of the control function 151 that the patient's intake status of the prescription medicine has deviated from the predetermined intake status by detecting that the patient failed to take the prescription medicine. Specifically, the medicine intake failure detection function 157 acquires the past medicine intake time, which is the time when the patient previously took the prescription medicine, among the medicine intake times stored in the medicine intake history database 114 of the memory circuitry 11 via the stored information acquisition function 156. The medicine intake failure detection function 157 also acquires the past scheduled medicine intake time, which is the time when the patient was previously scheduled to take the prescription medicine, among the scheduled medicine intake times stored in the medicine intake schedule database 113 of the memory circuitry 11 via the stored information acquisition function 156. Then, the medicine intake failure detection function 157 detects that the patient failed to take the prescription medicine at the past scheduled medicine intake time based on the past medicine intake time and the past scheduled medicine intake time. Here, “previous” refers to a “most recent past relative to the current time.”

The excessive medicine intake detection function 158 detects that the patient's intake status of the prescription medicine has deviated from the predetermined intake status by detecting that the patient has taken the prescription medicine excessively under the control of the control function 151. Specifically, the excessive medicine intake detection function 158 acquires at least one past medicine intake time including at least the past medicine intake time, among the medicine intake times stored in the stored in the medicine intake history database 114 of the memory circuitry 11 via the stored information acquisition function 156. The excessive medicine intake detection function 158 also acquires at least one past scheduled medicine intake time including at least the past scheduled medicine intake time, among the scheduled medicine intake times stored in the medicine intake schedule database 113 of the memory circuitry 11 via the stored information acquisition function 156. Then, the excessive medicine intake detection function 158 detects that the patient has taken the prescription medicine excessively based on the at least one past medicine intake time and the at least one past scheduled medicine intake time.

If the patient's intake status of the prescription medicine deviates from the predetermined intake status, the response method suggestion function 159 suggests a response method against the patient's intake status of the prescription medicine deviating from the predetermined intake status based on the properties of the prescription medicine, the past medicine intake time, and the next scheduled medicine intake time, which is a predetermined time when the patient will take a next dose of the prescription medicine. “Next” refers to a “closest future relative to the previous time.” The response method suggestion function 159 suggests the response method against the patient's intake status of the prescription medicine deviating from the predetermined intake status to the information terminal device 2 of the healthcare professional. The response method suggestion function 159 may also suggest the response method against the patient's intake status of the prescription medicine deviating from the predetermined intake status to the information terminal device 3 of the patient.

Specifically, if the patient failed to take the prescription medicine (i.e., did not take the medicine), the response method suggestion function 159 suggests the response method against the patient's intake status of the prescription medicine deviating from the predetermined intake status by suggesting a response method against the patient failing to take the prescription medicine (hereinafter, also referred to as response method against medicine intake failure) based on the measure against medicine intake failure, the past medicine intake time, the next scheduled medicine intake time, and the current time stored in the memory circuitry 11. Specifically, if the medicine intake failure detection function 157 detects that the patient failed to take the prescription medicine at the past scheduled medicine intake time, the response method suggestion function 159 acquires the measure against medicine intake failure and the properties of the prescription medicine stored in the prescription medicine information database 112 of the memory circuitry 11 via the stored information acquisition function 156. The response method suggestion function 159 also acquires the past medicine intake time, which is the time when the patient previously took the prescription medicine, among the medicine intake times stored in the medicine intake history database 114 of the memory circuitry 11 via the stored information acquisition function 156. The response method suggestion function 159 also acquires the next scheduled medicine intake time among the scheduled medicine intake times stored in the medicine intake schedule database 113 of the memory circuitry 11 via the stored information acquisition function 156. Then, the response method suggestion function 159 suggests the response method against medicine intake failure based on the acquired properties of the prescription medicine, the measure against medicine intake failure, the past medicine intake time, the next scheduled medicine intake time, and the current time.

Also, the response method suggestion function 159 suggests the response method against the patient's intake status of the prescription medicine deviating from the predetermined intake status by suggesting the response method against the patient taking the prescription medicine excessively (hereinafter, also referred to as the response method against excessive medicine intake). Specifically, if the excessive medicine intake detection function 158 detects that the patient has taken the prescription medicine excessively, the response method suggestion function 159 acquires the measure against excessive medicine intake and the properties of the prescription medicine stored in the prescription medicine information database 112 of the memory circuitry 11 via the stored information acquisition function 156. The response method suggestion function 159 also acquires the past medicine intake time, which is the time when the patient previously took the prescription medicine, among the medicine intake times stored in the medicine intake history database 114 of the memory circuitry 11 via the stored information acquisition function 156. The response method suggestion function 159 also acquires the next scheduled medicine intake time among the scheduled medicine intake times stored in the medicine intake schedule database 113 of the memory circuitry 11 via the stored information acquisition function 156. Then, the response method suggestion function 159 suggests the response method against excessive medicine intake based on the acquired properties of the prescription medicine, the measure against excessive medicine intake (i.e., a second measure), the past medicine intake time, the next scheduled medicine intake time, and the current time.

More specifically, the response method suggestion function 159 calculates a next recommended medicine intake time, which is the time recommended for the patient to take the next dose of the prescription medicine, based on the properties of the prescription medicine, the past medicine intake time, the next scheduled medicine intake time, and the current time. The response method suggestion function 159 suggests the response method against medicine intake failure and excessive medicine intake based on the calculated next recommended medicine intake time.

For instance, if the current time is calculated as the next recommended medicine intake time after medicine intake failure, the response method suggestion function 159 suggests as the response method against medicine intake failure, to take the prescription medicine, which was failed to be taken at the past scheduled medicine intake time, now. On the other hand, if the next scheduled medicine intake time is calculated as the next recommended medicine intake time after medicine intake failure, the response method suggestion function 159 suggests taking the prescription medicine, which was failed to be taken at the past scheduled medicine intake time, at the next scheduled medicine intake time without taking it now.

When detecting that the patient failed to take the prescription medicine, the medicine intake failure detection function 157 may issue an alarm of medicine intake failure to the information terminal device 3 of the patient and prompt the patient for a confirmation operation of medicine intake failure. When the confirmation operation of medicine intake failure is detected, the response method suggestion function 159 may predict the blood concentration of the prescription medicine at a time of detecting the confirmation operation of medicine intake failure and a maximum blood concentration of the prescription medicine in the case of taking the prescription medicine at both the current time and the next scheduled medicine intake time based on the properties of the prescription medicine. Then, the response method suggestion function 159 may determine whether to set either the current time or the next scheduled medicine intake time as the next recommended medicine intake time based on the blood concentration and the maximum blood concentration that were predicted.

If the next scheduled medicine intake time is calculated as the next recommended medicine intake time after excessive medicine intake, the response method suggestion function 159 suggests taking the prescription medicine at the next scheduled medicine intake time as the response method against excessive medicine intake. On the other hand, if a time after the next scheduled medicine intake time is calculated as the next recommended medicine intake time after excessive medicine intake, the response method suggestion function 159 suggests taking the prescription medicine on the time after the next scheduled medicine intake time without taking the prescription medicine at the next scheduled medicine intake time as the response method against excessive medicine intake.

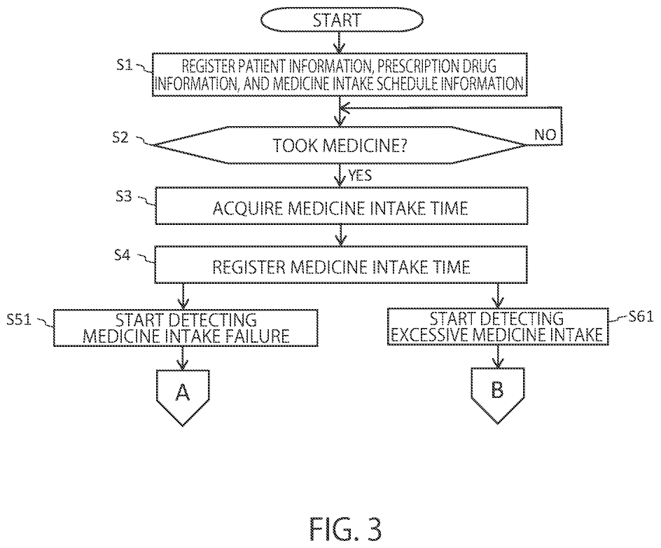

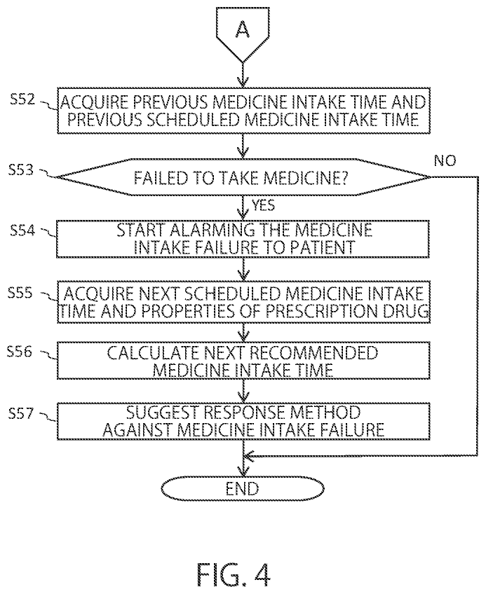

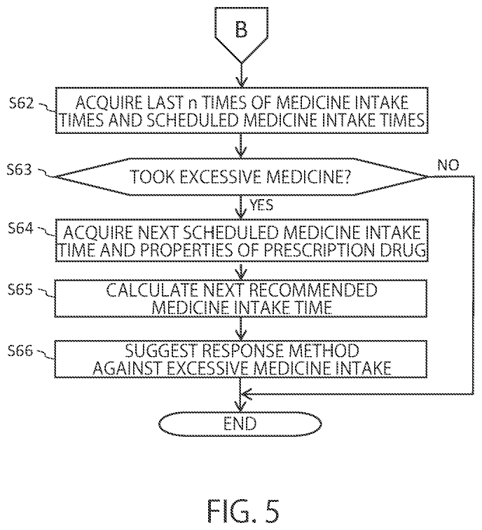

Next, an exemplary operation the medicine intake support device 10 according to the first embodiment configured as above will be described. FIG. 3 is a flowchart illustrating the exemplary operation of the medicine intake support device 10 according to the first embodiment. FIG. 4 is a flowchart continued from that of FIG. 3, illustrating the exemplary operation of the medicine intake support device 10 according to the first embodiment. FIG. 5 is a flowchart continued from that of FIG. 3, illustrating the exemplary operation of the medicine intake support device 10 according to the first embodiment.

First, as shown in FIG. 3, the basic information registration function 152 acquires the patient information, the prescription medicine information, and the medicine intake schedule information input from the input interface 12. The basic information registration function 152 registers the acquired patient information to the patient information database 111 of the memory circuitry 11. The basic information registration function 152 also registers the acquired prescription medicine information to the prescription medicine information database 112 of the memory circuitry 11. The basic information registration function 152 also registers the acquired medicine intake schedule information to the medicine intake schedule database 113 of the memory circuitry 11 (Step S1).

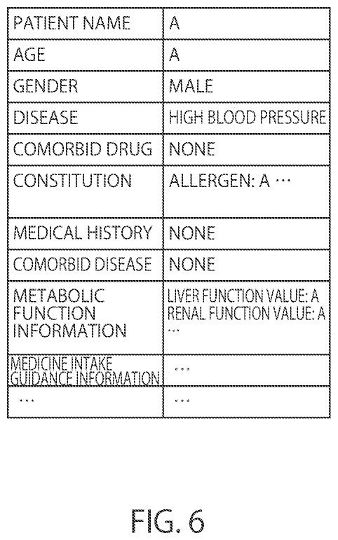

FIG. 6 is a description diagram for describing a registration process of the patient information in the exemplary operation of the medicine intake support device 10 according to the first embodiment. For instance, as shown in FIG. 6, the basic information registration function 152 registers patient information including a name of the patient, the age, a gender, the disease, a comorbid drugs, a constitution, the medical history, the comorbid disease, the metabolic function information, and the medicine intake guidance information to the patient information database 111.

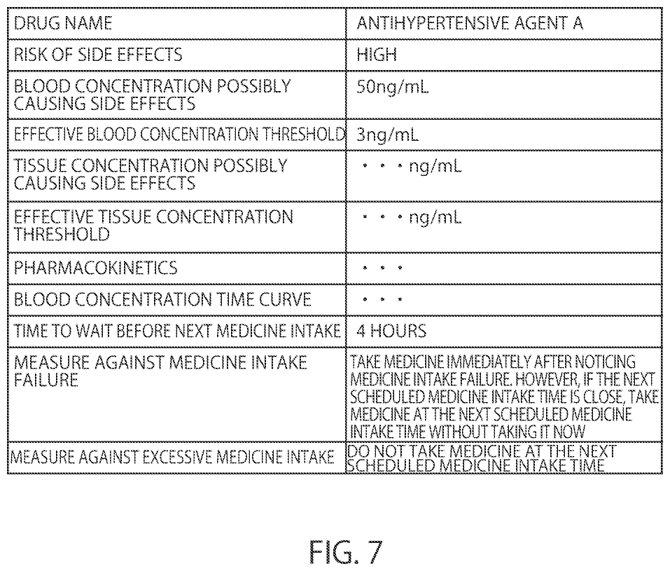

FIG. 7 is a description diagram for describing the registration process of the prescription medicine information in the exemplary operation of the medicine intake support device 10 according to the first embodiment. For instance, as shown in FIG. 7, the basic information registration function 152 registers the prescription medicine information including a drug name that also serves as the type of the prescription medicine, the properties of the prescription medicine, the measure against medicine intake failure, the measure against excessive medicine intake, and the time to wait before the next medicine intake, to the prescription medicine information database 112. The type of the prescription medicine may be registered to the prescription medicine information database 112 independently from the drug name. As shown in FIG. 7, the basic information registration function 152 may register the drug name, the severity of side effects, the blood concentration possibly causing the side effects, an effective blood concentration threshold, the tissue concentration possibly causing the side effects, an effective tissue concentration threshold, and a pharmacokinetics including the blood concentration time curve, to the prescription medicine information database 112 as the properties of the prescription medicine.

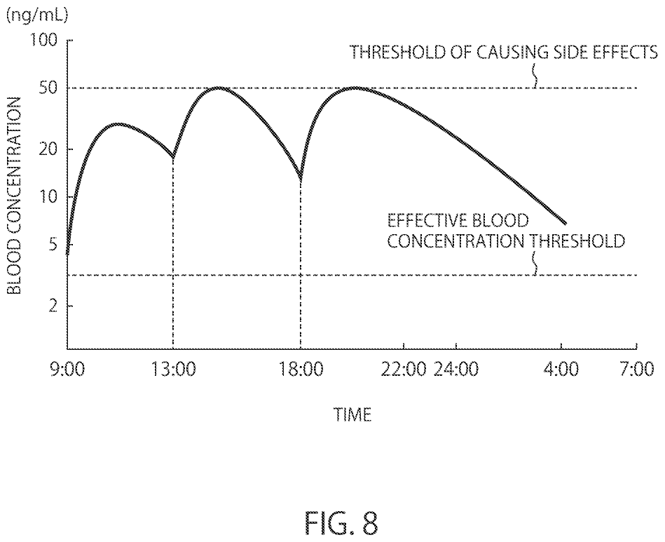

FIG. 8 is a description diagram different from FIG. 7, for describing the registration process of the prescription medicine information in the exemplary operation of the medicine intake support device 10 according to the first embodiment. The basic information registration function 152 may register the blood concentration time curve (pharmacokinetics) as shown in FIG. 8 to the prescription medicine information database 112. In the example shown in FIG. 8, the blood concentration time curve shows a time change of blood concentration of the antihypertensive agent A when the antihypertensive agent A was taken three times a day at the scheduled medicine intake times (9:00, 13:00, 18:00). In the example shown in FIG. 8, the effective blood concentration threshold is 3 ng/mL. The threshold possibly causing the side effects is 50 ng/mL.

The basic information registration function 152 may register the blood concentration time curve to the prescription medicine information database 112 following the input operation of the input interface 12. Or the basic information registration function 152 may predict the blood concentration time curve of FIG. 8 based on the patient information and the medicine intake schedule information and register the blood concentration time curve of FIG. 8 acquired by estimation to the prescription medicine information database 112. The blood concentration time curve acquired by estimation contains contents unique for each patient corresponding to the patient information and the medicine intake schedule information. For instance, even with the same dosage amount of the prescription medicine, the blood concentration may be different corresponding to a height or weight etc., of the patient. Thus, it becomes possible to suggest response methods against medicine intake failure and response methods against excessive medicine intake more adequately by predicting the blood concentration time curve made unique for each patient. Properties of the prescription medicine other than the blood concentration time curve may also be made unique for each patient and be registered based on the patient information and the medicine intake schedule information.

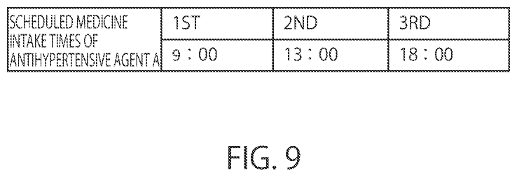

FIG. 9 is a description diagram for describing the registration process of the medicine intake schedule information in the exemplary operation of the medicine intake support device 10 according to the first embodiment. As shown in FIG. 9, the basic information registration function 152 may register the scheduled medicine intake times of the antihypertensive agent A three times a day (9:00, 13:00, 18:00) to the medicine intake schedule database 113 as the medicine intake schedule information.

After the patient information, the prescription medicine information, and the medicine intake schedule information are registered, as shown in FIG. 3, the medicine intake checking function 153, under the control of the control function 151, determines whether the patient took the prescription medicine (Step S2). For instance, the medicine intake checking function 153 may detect that the patient took the prescription medicine using the medicine intake storage system that detects and notifies that the prescription medicine has been removed from the storage container with sensors provided in the storage container. Or the medicine intake checking function 153 may detect that the patient took the prescription medicine using the detection system that detects intake behavior of medicine with the surveillance camera. Else, the medicine intake checking function 153 may detect that the prescription medicine was taken using the notification system that detects and notifies that the prescription medicine was taken with a sensor attached to the prescription medicine itself. The sensor may be configured to generate electrical signals as the prescription medicine melts inside a stomach. Or the medicine intake checking function 153 may detect that the prescription medicine was taken in response to the patient notifying a completion of taking the medicine (e.g., pushing an intake button on an operating screen) to the medicine intake support device 10 using the information terminal device 3 of the patient. If the intake of prescription medicine is detected with a method other than the notification of completing the intake from the patient, the medicine intake checking function 153 may send an inquiry to the information terminal device 3 of the patient of whether the medicine was taken to increase a certainty of the detection.

When detecting that the patient has taken the prescription medicine (Step S2: Yes), the medicine intake time acquisition function 154 acquires the time of detecting that the patient took the prescription medicine as the medicine intake time from a clock system not shown (Step S3).

On the other hand, when failing to detect that the patient has taken the prescription medicine (Step S2: No), the medicine intake checking function 153 repeats determining whether the patient has taken the prescription medicine (Step S2).

After the medicine intake time is acquired, the medicine intake time registration function 155 registers the acquired medicine intake time to the medicine intake history database 114 (Step S4).

After the medicine intake time is registered, the medicine intake failure detection function 157 starts detecting the medicine intake failure (Step S51) to support responding to the medicine intake failure. Also, the excessive medicine intake detection function 158 starts detecting excessive medicine intake (Step S61) to support responding to excessive medicine intake. The medicine intake failure detection function 157 may start detecting the medicine intake failure, for instance, after a predetermined time (e.g., 30 minutes) has passed from the scheduled medicine intake time. Also, the excessive medicine intake detection function 158 may start detecting excessive medicine intake, for instance, when the medicine intake time is newly registered.

First, a support responding to medicine intake failure will be described. After starting to detect the medicine intake failure, the stored information acquisition function 156 extracts and acquires the past medicine intake time from the medicine intake history database 114 of the memory circuitry 11. The stored information acquisition function 156 also extracts and acquires the past scheduled medicine intake time from the MHD 113 of the memory circuitry 11 (Step S52).

After the past medicine intake time and the past scheduled medicine intake time are acquired, the medicine intake failure detection function 157 determines the medicine intake failure based on the acquired past medicine intake time and the past scheduled medicine intake time (Step S53). Specifically, the medicine intake failure detection function 157 determines failure of medicine intake based on whether the past medicine intake time is within an allowable time before and after the past scheduled medicine intake time. In other words, the medicine intake failure detection function 157 determines that the medicine was taken if the past medicine intake time is within the allowable time before and after the past scheduled medicine intake time. On the other hand, the medicine intake failure detection function 157 determines that the medicine was not taken if the past medicine intake time is not within the allowable time before and after the past scheduled medicine intake time.

When determining that the medicine was not taken (Step S53: Yes), the medicine intake failure detection function 157 starts alarming the patient that the medicine was not taken (Step S54). Specifically, the medicine intake failure detection function 157 sends the alarm of medicine intake failure to the information terminal device 3 of the patient every arbitrary time (e.g., 10 minutes) until the patient notices. The alarm of medicine intake failure may be issued by displaying an operation button that prompts the confirmation operation of medicine intake failure to the patient on the screen of the information terminal device 3 of the patient. When detecting the confirmation operation of medicine intake failure by operating the operation button, the medicine intake failure detection function 157 stores the time of detecting the confirmation operation of medicine intake failure, i.e., the time when the patient notices medicine intake failure, to the memory circuitry 11. The medicine intake failure detection function 157 may also store the time when the patient notices the medicine intake failure to, for instance, the medicine intake history database 114 of the memory circuitry 11. The confirmation operation of medicine intake failure using the operation button may be performed by the patient without waiting for the alarm of medicine intake failure. Also, the alarm of medicine intake failure may include warning the patient to not take the medicine now because the scheduled medicine intake time has already passed. Also, in the case of drugs that may cause serious side effects, inquiries may be made to healthcare professionals such as family doctors and pharmacists. The inquiry of the healthcare professionals may be automatically performed by the medicine intake failure detection function 157. Or the inquiries may be manually performed using communication means by the patient such as a call or an email.

On the other hand, when determining that the medicine was taken (Step S53: No), the medicine intake failure detection function 157 terminates the process until reaching a new detection period.

Next, the response method suggestion function 159 extracts and acquires the next scheduled medicine intake time from the medicine intake schedule database 113 of the memory circuitry 11. The response method suggestion function 159 also extracts and acquires the properties of the prescription medicine from the prescription medicine information database 112 of the memory circuitry 11 (Step S55).

After the next scheduled medicine intake time and the properties of the prescription medicine are acquired, the response method suggestion function 159 calculates the next recommended medicine intake time based on the properties of the prescription medicine, the past medicine intake time, the next scheduled medicine intake time, and the current time (Step S56).

For instance, the response method suggestion function 159 predicts the blood concentration of the prescription medicine at the current time based on the properties of the prescription medicine. The response method suggestion function 159 also predicts the maximum blood concentration of the prescription in the case of taking the prescription medicine at both the current time and the next scheduled medicine intake time based on the properties of the prescription medicine. The response method suggestion function 159 also predicts a transition in blood concentration of the prescription medicine if the prescription medicine is not taken at the current time. The response method suggestion function 159 may predict the blood concentration of the prescription medicine at the time when the confirmation operation of medicine intake failure was detected, as the blood concentration of the prescription medicine at the current time. The response method suggestion function 159 may also predict the maximum blood concentration of the prescription medicine in the case of taking the prescription medicine at both the time of detecting the confirmation operation of medicine intake failure and the next scheduled medicine intake time, as the maximum blood concentration of the prescription medicine in the case of taking the prescription medicine at both the current time and the next scheduled medicine intake time. The response method suggestion function 159 may also predict the transition in blood concentration of the prescription medicine in the case of taking no medicine at the time when the confirmation operation of medicine intake failure was detected, as the transition in blood concentration of the prescription medicine in the case of taking no medicine at the current time. Then, the response method suggestion function 159 determines whether to set either the current time or the next scheduled medicine intake time as the next recommended medicine intake time based on the blood concentration of the prescription medicine at the current time, the maximum blood concentration of the prescription medicine in the case of taking the prescription medicine at both the current time and the next scheduled medicine intake time, and the transition in blood concentration of the prescription medicine in the case of taking no medicine at the current time.

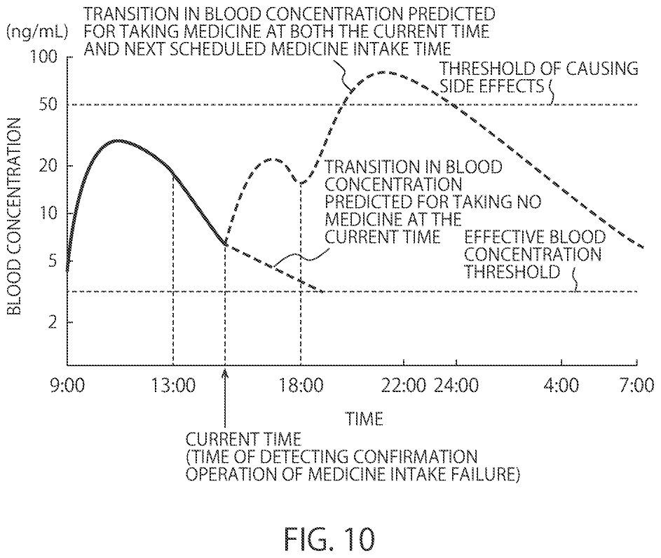

FIG. 10 is a description diagram for describing a calculating process of the next recommended medicine intake time in the exemplary operation of the medicine intake support device 10 according to the first embodiment. In the example shown in FIG. 10, the response method suggestion function 159 predicts the blood concentration of the antihypertensive agent A at the current time (the time when the confirmation operation of medicine intake failure was detected), the maximum blood concentration of the antihypertensive agent A in the case of taking the prescription medicine at both the current time and the next scheduled medicine intake time, and the transition in blood concentration of the prescription medicine of the antihypertensive agent A in the case of taking no medicine at the current time, based on the blood concentration time curve of the antihypertensive agent A. Specifically, the response method suggestion function 159 predicts that the blood concentration of the antihypertensive agent A on 15:00, which is the current time (the time when the confirmation operation of medicine intake failure was detected) is 7 ng/mL. The response method suggestion function 159 also predicts that the maximum blood concentration in the case of taking the medicine on both 15:00, which is the current time, and 18:00, which is the next scheduled medicine intake time, is 90 ng/mL. Such high maximum blood concentration originates from a time interval from 15:00, which is the current time, to 18:00, which is the next scheduled medicine intake time, being shorter than 4 hours (refer to FIG. 7) provided as the time to wait before the next dose. Then, the response method suggestion function 159 detects that the maximum blood concentration 90 ng/mL exceeds the blood concentration threshold of antihypertensive agent A 50 ng/mL that may possibly cause side effects. On the other hand, when predicting that the transition in blood concentration of the antihypertensive agent A in the case of taking no medicine at the current time, the response method suggestion function 159 detects that the blood concentration of the antihypertensive agent A does not fall below the effective blood concentration threshold 3 ng/mL. By such, the response method suggestion function 159 determines that the next recommended medicine intake time is the next scheduled medicine intake time (18:00).

Although the next recommended medicine intake time was determined to be the next scheduled medicine intake time in the example shown in FIG. 10, the next recommended medicine intake time may be determined to be the current time depending on the prediction results of the response method suggestion function 159. The response method suggestion function 159 may also calculate the next recommended medicine intake time and shift back the recommended medicine intake times after the next dose. In this case, the recommended medicine intake times after the next dose may gradually be returned to the scheduled medicine intake times as usual by adjusting a time width of shifting back the recommended medicine intake times after the next dose. Further, there may be cases where the next recommended medicine intake time may be calculated with a calculating method more complex than determining the next recommended medicine intake time as either the current time or the next scheduled medicine intake time, depending on a combination of comorbid drugs and a degree of overlapping components. In such case, the next recommended medicine intake time may be calculated with high precision, using, for instance, a trained model from machine learning of correlation between inputs and outputs where the medicine intake time of the prescription medicine, the scheduled medicine intake time of the prescription medicine, the medicine intake time of the comorbid drug, and the like, are the inputs and the next recommended medicine intake time is the output. Also, although the response method suggestion function 159 calculated the next recommended medicine intake time using the blood concentration time curve of the antihypertensive agent A in the example shown in FIG. 10, configurations are not limited to such, and for instance, the response method suggestion function 159 may calculate the next recommended medicine intake time using a tissue concentration time curve of the antihypertensive agent A.

After calculating the next recommended medicine intake time, the response method suggestion function 159 suggests the response method against medicine intake failure as shown in FIG. 4 (Step S57). Specifically, if the current time is calculated as the next recommended medicine intake time, the response method suggestion function 159 suggests taking the prescription medicine, which was missed at the past scheduled medicine intake time, now as the response method against medicine intake failure. On the other hand, if the next scheduled medicine intake time is calculated as the next recommended medicine intake time, the response method suggestion function 159 suggests taking the prescription medicine, which was missed at the past scheduled medicine intake time, at the next scheduled medicine intake time without taking it now as the response method against medicine intake failure. The response method against medicine intake failure suggested by the response method suggestion function 159 is information that further limits (i.e., specifies) the contents of the measure against medicine intake failure stored in the prescription medicine information database 112 in response to the intake status. The response method suggestion function 159 may also suggest the measure against medicine intake failure stored in the prescription medicine information database 112.

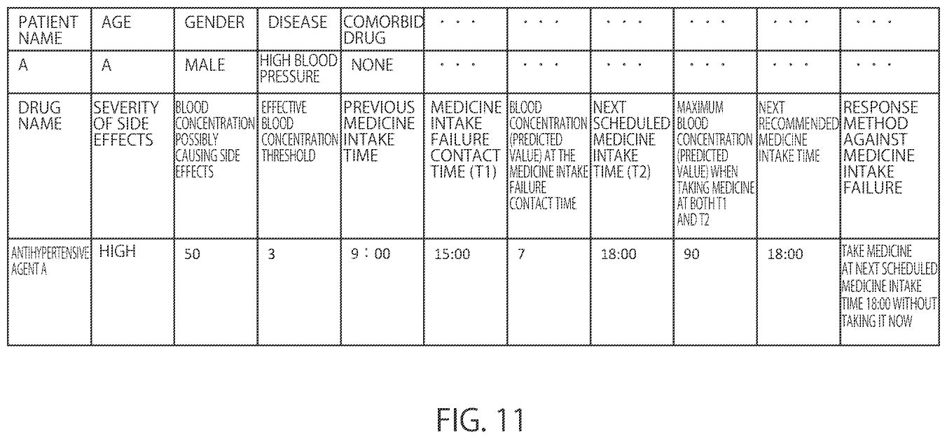

FIG. 11 is a description diagram for describing a suggestion process of the response method against medicine intake failure in the exemplary operation of the medicine intake support device 10 according to the first embodiment. In the example shown in FIG. 11, the response method suggestion function 159 sends a response method suggestion image for medicine intake failure shown in FIG. 11 to the information terminal device 2 of the healthcare professional. The response method suggestion image for medicine intake failure describes a medicine intake failure contact time T1, which is an example of the current time, the blood concentration when contacting medicine intake failure, the maximum blood concentration in the case of taking the medicine at both the medicine intake failure contact time and the next scheduled medicine intake time, the next recommended medicine intake time, and the response method against medicine intake failure, in addition to the patient information, properties of the prescription medicine (antihypertensive agent A), the past medicine intake time, and the next scheduled medicine intake time. T1 is the time when the confirmation operation of medicine intake failure was detected. In the example shown in FIG. 11, the response method against medicine intake failure is to take the medicine on 18:00, which is the next scheduled medicine intake time, without taking it now.

By displaying the response method suggestion image for medicine intake failure as shown in FIG. 11 on the information terminal device 2 of the healthcare professional, the healthcare professional may conveniently and adequately determine the response method against medicine intake failure. By such, the healthcare professional may use the communication means such as the call or email to perform medicine intake guidance for medicine intake failure of the patient quickly and adequately. The medicine intake guidance for medicine intake failure of the patient may be performed via the information terminal device 3 of the patient. Further, the response method suggestion function 159 may suggest the response method against medicine intake failure to the information terminal device 3 of the patient for prescription medicines determined in advance to have low severity of side effects by the healthcare professional.

Next, the response method against excessive medicine intake will be described. After starting to detect excessive medicine intake in FIG. 3, as shown in FIG. 5, the stored information acquisition function 156 extracts and acquires the last n times (hereinafter, n is an arbitrary natural number) of medicine intake times from the medicine intake history database 114 of the memory circuitry 11. The stored information acquisition function 156 also extracts and acquires the last n times of scheduled medicine intake times from the medicine intake schedule database 113 of the memory circuitry 11 (Step S62).

After the last n times of medicine intake times and the last n times of scheduled medicine intake times are acquired, the excessive medicine intake detection function 158 determines whether there was excessive medicine intake based on the acquired last n times of medicine intake times and the last n times of scheduled medicine intake times (Step S63). Specifically, the excessive medicine intake detection function 158 compares the last n times of medicine intake times with the last n times of scheduled medicine intake times. Then, the excessive medicine intake detection function 158 determines whether the last n times of medicine intake times match with the last n times of scheduled medicine intake times based on the allowed time of a difference of the medicine intake times from the scheduled medicine intake times. If the last n times of medicine intake times match with the last n times of scheduled medicine intake times, the excessive medicine intake detection function 158 determines that there is no excessive medicine intake. On the other hand, if there is a non-matching combination between the last n times of medicine intake times and the last n times of scheduled medicine intake times, for instance, if the past scheduled medicine intake time matches with the medicine intake time before the past medicine intake time instead of the past medicine intake time, the excessive medicine intake detection function 158 determines that there is excessive medicine intake. By such, the excessive medicine intake detection function 158 detects excessive medicine intake. The excessive medicine intake detection function 158 may further detect excessive medicine intake by receiving a contact of excessive medicine intake from the patient through the information terminal device 3 of the patient or the like.

When determining that there was excessive medicine intake (Step S63: Yes), the response method suggestion function 159 extracts and acquires the next scheduled medicine intake time from the medicine intake schedule database 113 of the memory circuitry 11 via the stored information acquisition function 156. The response method suggestion function 159 also extracts and acquires the properties of the prescription medicine from the prescription medicine information database 112 of the memory circuitry 11 via the stored information acquisition function 156.

On the other hand, when determining that there was no excessive medicine intake (Step S63: No), the excessive medicine intake detection function 158 terminates the process until reaching the new detection period.

After acquiring the next scheduled medicine intake time and the properties of the prescription medicine, the response method suggestion function 159 calculates the next recommended medicine intake time based on the properties of the prescription medicine, the past medicine intake time, the next scheduled medicine intake time, and the current time (Step S65). For instance, the response method suggestion function 159 may predict the blood concentration of the antihypertensive agent A on 10:00, which is the time when the excessive medicine intake was detected. The response method suggestion function 159 also predicts the maximum blood concentration after the next scheduled medicine intake time (13:00) in the case of taking the medicine at both the time of detecting excessive medicine intake (10:00) and 13:00, which is the next scheduled medicine intake time. In this example, the response method suggestion function 159 predicts that the blood concentration of the antihypertensive agent A is 90 ng/mL at the time of detecting excessive medicine intake (10:00). The response method suggestion function 159 also predicts that the maximum blood concentration after the next scheduled medicine intake time (13:00) is 50 ng/mL in the case of taking the antihypertensive agent A at both the time of detecting excessive medicine intake (10:00) and the next scheduled medicine intake time (13:00). By such, the response method suggestion function 159 detects that the maximum blood concentration after the next scheduled medicine intake time (13:00) reaches the threshold of the antihypertensive agent A that possibly causes side effects 50 ng/mL. This originates from the time interval from the time of detecting excessive medicine intake (10:00) to the next scheduled medicine intake time (13:00) being shorter than 4 hours (refer to FIG. 7) provided as the time to wait before the next dose. In this case, the response method suggestion function 159 calculates 15:00, which is the time where a time greater than four hours (five hours) has past from the time of detecting excessive medicine intake (10:00), as the next recommended medicine intake time. The response method suggestion function 159, in addition to the next recommended medicine intake time, may further calculate the recommended medicine intake times after the next dose. For instance, the response method suggestion function 159 may calculate 19:00, four hours later the next recommended medicine intake time (15:00), as the recommended medicine intake time after the next dose.

Further, the next scheduled medicine intake time may be calculated as the next recommended medicine intake time depending on the predictions of the response method suggestion function 159. Also, similar to supporting response to medicine intake failure, the next recommended medicine intake time may be calculated with the complex calculation method depending on the combination of the comorbid drugs or the degree of overlapping components. In this case, similar to supporting response to medicine intake failure, the next recommended medicine intake time may be calculated with high accuracy using the trained model from machine learning of correlation between inputs and outputs where the medicine intake time of the prescription medicine, the scheduled medicine intake time of the prescription medicine, and the medicine intake time of the comorbid drug, etc., are the inputs and the next recommended medicine intake time is the output.

As shown in FIG. 5, after calculating the next recommended medicine intake time, the response method suggestion function 159 suggests the response method against excessive medicine intake (Step S66). Specifically, if the next scheduled medicine intake time is calculated as the next recommended medicine intake time, the response method suggestion function 159 suggests taking the medicine at the next scheduled medicine intake time, as the response method against excessive medicine intake. On the other hand, if a time after the next scheduled medicine intake time is calculated as the next recommended medicine intake time, the response method suggestion function 159 suggests taking the prescription medicine at the time after the next scheduled medicine intake time without taking the prescription medicine at the next scheduled medicine intake time, as the response method against excessive medicine intake. The response method against excessive medicine intake suggested by the response method suggestion function 159 is information that further limits (specifies) the contents of the measure against excessive medicine intake stored in the prescription medicine information database 112 in response to the medicine intake status. The response method suggestion function 159 may further suggest the measure against excessive medicine intake stored in the prescription medicine information database 112.

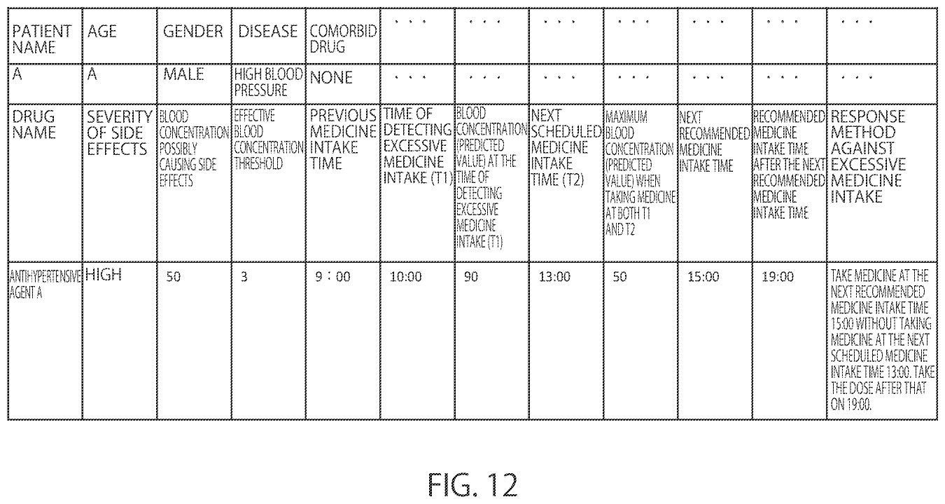

FIG. 12 is a description diagram for describing the suggestion process of the response method against excessive medicine intake in the exemplary operation of the medicine intake support device 10 according to the first embodiment. In the example shown in FIG. 12, the response method suggestion function 159 sends the response method suggestion image for excessive medicine intake shown in FIG. 12 to the information terminal device 2 of the healthcare professional. The response method suggestion image for excessive medicine intake describes the time of detecting excessive medicine intake T1, the blood concentration at T1, the maximum blood concentration after T2 in the case of taking the medicine at both T1 and T2, the next recommended medicine intake time, the recommended medicine intake time after the next dose, and the response method against excessive medicine intake, in addition to the patient information, properties of the prescription medicine (antihypertensive agent A), the past medicine intake time, and the next scheduled medicine intake time T2. In the example shown in FIG. 12, the response method against excessive medicine intake is to take the medicine at the next recommended medicine intake time (15:00) without taking at the next scheduled medicine intake time (13:00) and to take the dose after that on 19:00.

By displaying the response method against excessive medicine intake as shown in FIG. 12 to the information terminal device 2 of the healthcare professional, the healthcare professional may conveniently and adequately determine the response method against excessive medicine intake. By such, the healthcare professional may perform medicine intake guidance for excessive medicine intake of the patient quickly and adequately using communication means such as the call or email. The medicine intake guidance for excessive medicine intake of the patient may be performed via the information terminal device 3 of the patient. The response method suggestion function 159 may further suggest the response method against excessive medicine intake to the information terminal device 3 of the patient regarding prescription medicines determined in advance to have low severity of side effects by the healthcare professional.

An example using antihypertensives as the prescription medicine was described till now, but the first embodiment may also be applied to prescription medicines such as insulin preparations or anticancer agents. This is the same for other embodiments which will be described below.

As described above, in the first embodiment, the stored information acquisition function 156 acquires the properties of the prescription medicine, the past medicine intake time, and the next scheduled medicine intake time. Also, the memory circuitry 11 stores the type of the prescription medicine and the measure against medicine intake failure in correspondence with each other. Also, when the patient's intake status of the prescription medicine deviates from the predetermined intake status, the response method suggestion function 159 suggests the response method against the patient's intake status of the prescription medicine deviating from the predetermined intake status based on the properties of the prescription medicine, the measure against medicine intake failure, the past medicine intake time, the next scheduled medicine intake time, and the current time.

By such, it becomes possible to support adequate response methods for the patient when the patient failed to take the prescribed medicine.

Also, in the first embodiment, the stored information acquisition function 156 acquires at least one of the severity of side effects of the prescription medicine, the likelihood of side effects of the prescription medicine, the effective blood concentration of the prescription medicine, and the effective tissue concentration of the prescription medicine as the properties of the prescription medicine.

By such, it becomes possible to suggest response methods for when the patient failed to take the prescribed medicine more adequately.

Also, in the first embodiment, the stored information acquisition function 156 acquires at least one of the blood concentration time curve showing the time change in blood concentration of the prescription medicine, the blood concentration of the prescription medicine that possibly causes side effects, and the tissue concentration of the prescription medicine that possibly causes side effects.

By such, it becomes possible to suggest response methods for when the patient failed to take the prescribed medicine further adequately.

Also, in the first embodiment, the stored information acquisition function 156 acquires the properties of the prescription medicine with contents corresponding to the characteristics of the patient.

By such, the response methods for when the patient failed to take the prescribed medicine may be suggested with more adequate contents made unique for each patient.

Also, in the first embodiment, the stored information acquisition function 156 acquires the properties of the prescription medicine corresponding to at least one of the age of the patient, the constitution of the patient, and the drugs comorbid to the prescription medicine of the patient, as the properties of the prescription medicine of the contents corresponding to the characteristics of the patient.

By such, the response methods for when the patient failed to take the prescribed medicine may be suggested with more adequate contents further unique for each patient.

Also, in the first embodiment, the medicine intake checking function 153 detects that the patient took the prescription medicine. The stored information acquisition function 156 acquires the time of the patient taking the prescription medicine as the past medicine intake time by the medicine intake checking function 153.

By such, the past medicine intake time may be conveniently acquired.

Also, in the first embodiment, the response method suggestion function 159 calculates the next recommended medicine intake time based on the properties of the prescription medicine, the past medicine intake time, the next scheduled medicine intake time, and the current time, and suggests response methods based on the calculated next recommended medicine intake time.

By such, the response methods for when the patient failed to take the prescribed medicine may be suggested more adequately using the next recommended medicine intake time.

Also, in the first embodiment, the response method suggestion function 159 suggests the response methods against at least one of the information terminal devices 2, 3 of the healthcare professional and the patient.

By such, the response methods for when the patient failed to take the prescribed medicine may be quickly suggested via the information terminal devices 2, 3 of the healthcare professional and the patient.

Also, in the first embodiment, the stored information acquisition function 156 further acquires the past scheduled medicine intake time, which is the time when the patient was scheduled to take the prescription medicine. Also, the medicine intake failure detection function 157 and the excessive medicine intake detection function 158 detects that the patient failed to take the prescription medicine or has taken the prescription medicine excessively based on the past medicine intake time and the past scheduled medicine intake time. Then, the response method suggestion function 159 suggests the response methods when detecting the medicine intake failure or excessive medicine intake by the medicine intake failure detection function 157 or the excessive medicine intake detection function 158.

By such, it becomes possible to suggest response methods while precisely detecting the patient's medicine intake failure or excessive medicine intake.

Also, in the first embodiment, the medicine intake failure detection function 157 detects that the patient forgot to take the prescription medicine at the past scheduled medicine intake time based on the past medicine intake time and the past scheduled medicine intake time.

By such, it becomes possible to suggest the response method against medicine intake failure while precisely detecting the medicine intake failure.

Also, in the first embodiment, if the current time is calculated as the next recommended medicine intake time, the response method suggestion function 159 suggests taking the prescription medicine, which was missed at the past scheduled medicine intake time, now, as the response method. On the other hand, if the next scheduled medicine intake time is calculated as the next recommended medicine intake time, the response method suggestion function 159 suggests taking the prescription medicine, which was missed at the past scheduled medicine intake time, at the next scheduled medicine intake time without taking it now, as the response method.

By such, the response method against medicine intake failure may be suggested more adequately.

Also, in the first embodiment, when detecting that the patient failed to take the prescription medicine, the medicine intake failure detection function 157 issues the alarm of medicine intake failure to the information terminal device 3 of the patient and prompts the patient for the confirmation operation of medicine intake failure. Also, when detecting the confirmation operation of medicine intake failure, the response method suggestion function 159 predicts the blood concentration of the prescription medicine at the time of detecting the confirmation operation of medicine intake failure and the maximum blood concentration of the prescription medicine in the case of taking the prescription medicine at both the current time and the next scheduled medicine intake time, based on the properties of the prescription medicine. Then, the response method suggestion function 159 determines whether to set either the current time or the next scheduled medicine intake time as the next recommended medicine intake time based on the predicted blood concentration and the maximum blood concentration.

By such, the next recommended medicine intake time may be calculated conveniently and quickly.

Also, in the first embodiment, the stored information acquisition function 156 acquires at least one past medicine intake time including at least the past medicine intake time and the at least one past scheduled medicine intake time including at least the past scheduled medicine intake time. Then, the excessive medicine intake detection function 158 detects that the patient has taken the prescription medicine excessively based on the at least one past medicine intake time and the at least one past scheduled medicine intake time.

By such, it becomes possible to suggest response methods against excessive medicine intake while precisely detecting the excessive medicine intake.

Also, in the first embodiment, if the time after the next scheduled medicine intake time is calculated as the next recommended medicine intake time, the response method suggestion function 159 suggests taking the prescription medicine at the time after the next scheduled medicine intake time without taking the prescription medicine at the next scheduled medicine intake time, as the measure.

By such, response methods against excessive medicine intake may be suggested more adequately.

### Second Embodiment

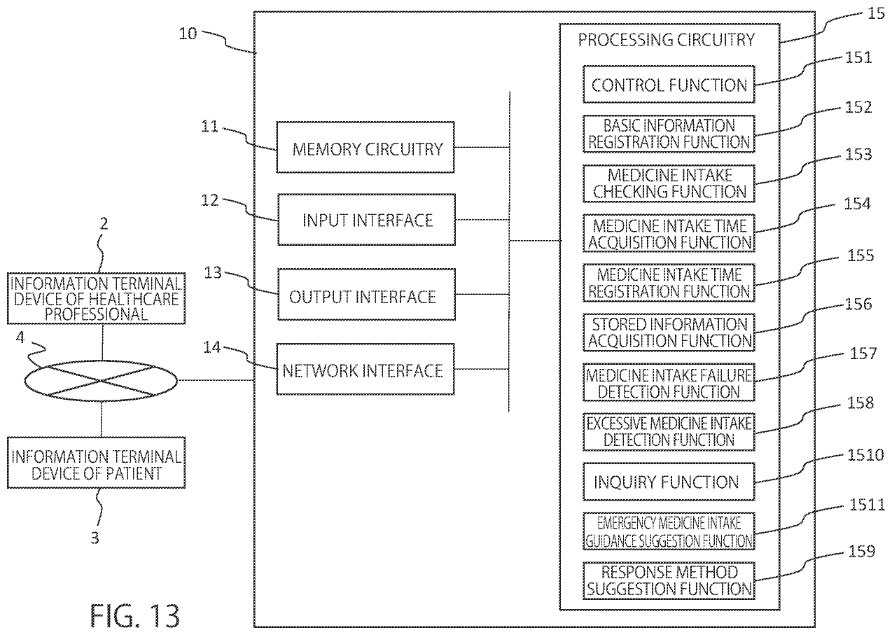

Next, in the case of detecting excessive medicine intake, a second embodiment that inquires an urgent medicine intake guidance will be described. FIG. 13 is a block diagram illustrating an exemplary configuration of the medicine intake support device 10 according to a second embodiment.

As shown in FIG. 13, the processing circuitry 15 according to the second embodiment further includes an inquiry function 1510 and an urgent medicine intake guidance suggestion function 1511 in addition to the configuration of the first embodiment. When the excessive medicine intake detection function 158 detects that the patient took the prescription medicine excessively, the inquiry function 1510 inquires of the healthcare professional for the urgent medicine intake guidance against the patient taking the prescription medicine excessively. The urgent medicine intake guidance suggestion function 1511 suggests the urgent medicine intake guidance responded by the healthcare professional to the patient.

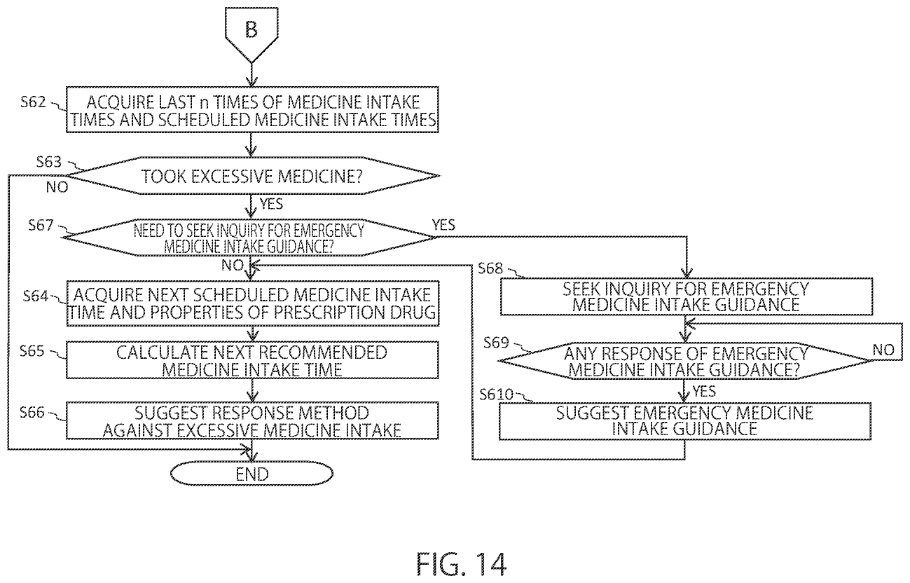

The exemplary operation of the medicine intake support device 10 according to the second embodiment will be mainly described with respect to differences from the first embodiment. FIG. 14 is a flowchart illustrating the exemplary operation of the medicine intake support device 10 according to the second embodiment. In the second embodiment, as shown in FIG. 14, when the excessive medicine intake detection function 158 detects that there was excessive medicine intake (Step S63: Yes), the inquiry function 1510 determines whether to inquire for the urgent medicine intake guidance (Step S67). Specifically, the inquiry function 1510 determines that the inquiry for the urgent medicine intake guidance is necessary if the severity of side effects in the properties of the prescription medicine is “high”. For instance, urgent medicine intake guidance may be required to point out the need for self-management since the excessive medicine intake of the antihypertensive agent may cause high blood pressure or bradycardia symptoms. Also, urgent medicine intake guidance may be required to point out the need for self-management since the excessive medicine intake of anticancer agents may greatly affect the constitution of the patient. On the other hand, the inquiry function 1510 determines that no inquiry for the urgent medicine intake guidance is necessary if the severity of side effects is “low”.

When determining that the inquiry for the urgent medicine intake guidance is necessary (Step S67: Yes), the inquiry function 1510 inquires of the urgent medicine intake guidance to the information terminal device 2 of the healthcare professional (Step S68). If the medicine intake support device 10 is installed in a medical institution where the healthcare professional in charge of the patient belongs to, the inquiry function 1510 may output the inquiry for the urgent medicine intake guidance from the output interface 13 to the healthcare professional.

On the other hand, when detecting that no inquiry for the urgent medicine intake guidance is necessary (Step S67: No), the inquiry function 1510 does not inquire for the urgent medicine intake guidance. In this case, a series of process to suggest response methods against excessive medicine intake (Step S64-Step S66) is executed.

After inquiring for the urgent medicine intake guidance, the inquiry function 1510 determines whether there is the response of the urgent medicine intake guidance for the inquiry.

If there is the response of the urgent medicine intake guidance (Step S69: Yes), the urgent medicine intake guidance suggestion function 1511 suggests the urgent medicine intake guidance to the information terminal device 3 of the patient (Step S610) and then proceeds to Step S64.

On the other hand, if there is no response of the urgent medicine intake guidance (Step S69: No), the inquiry function 1510 repeats determining whether there is the response of the urgent medicine intake guidance to the inquiry (Step S69). Further, if there is no response to the urgent medicine intake guidance (Step S69), the inquiry function 1510 may also repeat inquiring for the urgent medicine intake guidance (Step S68).

Further, the inquiry function 1510 may directly receive the inquiry from the patient via the information terminal device 3 of the patient and send the inquiry received from the patient to the healthcare professional. Also in this case, the urgent medicine intake guidance suggestion function 1511 may suggest the urgent medicine intake guidance responded by the healthcare professional to the patient.

As described above, in the second embodiment, if the excessive medicine intake detection function 158 detects the excessive medicine intake of the patient, the inquiry function 1510 inquires of the healthcare professional for the urgent medicine intake guidance to the excessive medicine intake corresponding to the properties of the prescription medicine. Also, the urgent medicine intake guidance suggestion function 1511 suggests the urgent medicine intake guidance responded by the healthcare professional to the patient.

By such, it becomes possible to suggest more adequate response methods against excessive medicine intake.

### Third Embodiment

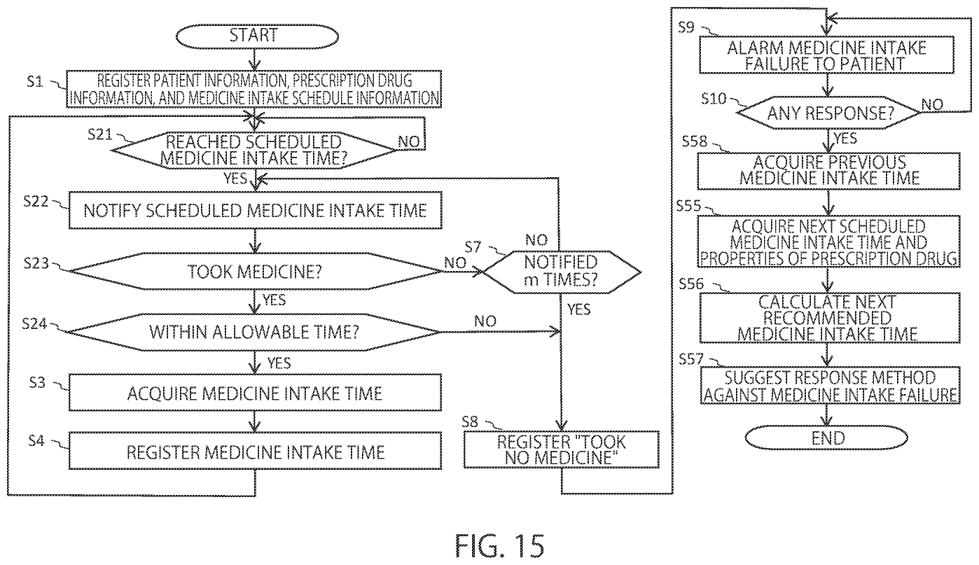

Next, a third embodiment that controls medicine intake failure will be described. Basic configurations of the medicine intake support device 10 according to the third embodiment is similar to that of the first embodiment (FIG. 1). FIG. 15 is a flowchart illustrating the exemplary operation of the medicine intake support device 10 according to the third embodiment.

As shown in FIG. 15, in the third embodiment, after registering the patient information, the prescription medicine information, and the medicine intake schedule information (Step S1), the medicine intake checking function 153 determines whether the scheduled medicine intake time has arrived (Step S21). Specifically, the medicine intake checking function 153 determines whether the scheduled medicine intake time has arrived based on the medicine intake schedule information stored in the medicine intake schedule database 113 of the memory circuitry 11.

If the scheduled medicine intake time has arrived (Step S21: Yes), the medicine intake checking function 153 notifies the scheduled medicine intake information to the information terminal device 3 of the patient (Step S22). It becomes possible to prompt the patient to take the prescription medicine by notifying the scheduled medicine intake information. Further, the notification of the scheduled medicine intake time may include a message that prompts to take the medicine.

On the other hand, if the scheduled medicine intake time has not arrived (Step S21: No), the medicine intake checking function 153 repeats determining whether the scheduled medicine intake time has arrived (Step S21).

After notifying the scheduled medicine intake time, the medicine intake checking function 153 determines whether the patient has taken the prescription medicine (Step S23). For instance, the medicine intake checking function 153 may detect the intake with means such as the intake storage, the surveillance camera, or the sensor attached to the prescription medicine itself described above.

If the patient took the prescription medicine (Step S23: Yes), the medicine intake checking function 153 determines whether the time when the patient took the prescription medicine is within the allowable time from the scheduled medicine intake time (Step S24).

On the other hand, if the patient did not take the prescription medicine (Step S23: No), the medicine intake checking function 153 determines whether a number of issuing the notification of the scheduled medicine intake time has reached m times (Step S7). m may be a predetermined fixed value or a variable value that may be arbitrarily set by the input operation via the input interface 12.

If the time when the patient took the prescription medicine is within the allowable time from the scheduled medicine intake time (Step S24: Yes), the medicine intake time acquisition function 154 acquires the time when the patient took the prescription medicine as the medicine intake time (Step S3).

On the other hand, if the time when the patient took the prescription medicine is not within the allowable time from the scheduled medicine intake time (Step S24: No), the medicine intake time acquisition function 154 registers “took no medicine” to the medicine intake history database 114 of the memory circuitry 11 (Step S8).

After acquiring the medicine intake time, the medicine intake time registration function 155 registers the acquired medicine intake time to the medicine intake history database 114 (Step S4).

If the number of issuing the notification has reached m times (Step S7: Yes), the medicine intake time acquisition function 154 registers “took no medicine” to the medicine intake history database 114 of the memory circuitry 11 (Step S8).

On the other hand, if the number of issuing the notification time has not reached m times (Step S7: No), the medicine intake checking function 153 repeats the notification of the scheduled medicine intake time to the information terminal device 3 of the patient (Step S22).

After “took no medicine” is registered, the medicine intake failure detection function 157 warns the patient of medicine intake failure (Step S9). Specifically, the medicine intake failure detection function 157 sends the alarm of medicine intake failure to the information terminal device 3 of the patient, repeating the alarm every arbitrary time (e.g., 10 minutes) until the patient notices. Specific embodiments of the alarm of medicine intake failure may be equivalent to that of the first embodiment (Step S54 of FIG. 4).

After sending the alarm of medicine intake failure for the patient, the medicine intake failure detection function 157 determines whether the patient has responded to the alarm (Step S10). Specifically, the medicine intake failure detection function 157 determines whether the patient has responded to the alarm based on whether the confirmation operation of medicine intake failure by the operation button described in FIG. 4 of the first embodiment was detected.

If the patient has responded to the alarm (Step S10: Yes), the medicine intake failure detection function 157 stores the time when the patient responded to the alarm, i.e., the time when the patient noticed to the medicine intake failure, to the memory circuitry 11. Then, the stored information acquisition function 156 extracts and acquires the past medicine intake time from the medicine intake history database 114 of the memory circuitry 11 (Step S58).

On the other hand, if the patient has not responded to the alarm (Step S10: No), the medicine intake failure detection function 157 repeats the alarm of medicine intake failure to the patient (Step S9).

After acquiring the past medicine intake time, the response method against medicine intake failure is suggested by executing Steps S55-S57 described in the first embodiment.

Further, a series of process to support responding to excessive medicine intake shown in Steps S2-S61 of FIG. 3 and Steps S62-S66 of FIG. 5 may be further executed in addition to each process shown in FIG. 15.

In the third embodiment, the medicine intake checking function 153 prompts the patient to take the medicine by repeatedly notifying the scheduled medicine intake time to the patient.

By such, it becomes able to control medicine intake failure. Also, it becomes possible to adequately suggest the response method against medicine intake failure if the medicine was not taken.

Note that the word “processor” used in above descriptions means circuits such as, for example, a Central Processing Unit (CPU), a Graphics Processing Unit (GPU), an Application Specific Integrated Circuitry (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory. Note that programs may be configured to be directly integrated in the processor instead of being stored in the memory. In this case, the processor realizes functions by reading and executing programs stored in the circuitry. Note that the processor is not limited to the case arranged as a single processor circuitry, but may be configured as a single processor by combining a plurality of independent circuits to realize functions. Furthermore, a plurality of component elements may be integrated into one processor to realize the functions.

According to at least one embodiment described above, it becomes possible to let the patient adequately respond if the patient's intake status deviates from the predetermined intake status.

While certain embodiments have been described, these embodiments have been presented by way of example only and are not intended to limit the scope of the inventions. The novel devices and methods described in the present disclosure may be in a variety of other forms. Furthermore, various omissions, substitutions and changes may be made for the embodiments of the devices and method of described in the present disclosure without departing from the spirit of the inventions. The embodiments and their modifications are included in the scope and the subject matter of the invention, and at the same time included in the scope of the claimed inventions and their equivalents.

What is claimed is:

1. A medicine intake support device, comprising:
processing circuitry configured to acquire a previous medicine intake time and a next scheduled medicine intake time, wherein the previous medicine intake time is a time when a patient previously took a prescription medicine, and the next scheduled medicine intake time is a predetermined time when the patient will take a next dose of the prescription medicine; and
a memory that stores a type of the prescription medicine and a measure in response to the patient's failure to take the type of the prescription medicine in correspondence with each other, wherein
the processing circuitry is further configured to, if the patient failed to take the prescription medicine, suggest a response method in response to the patient's failure to take the prescription medicine based on the measure stored in the memory, the previous medicine intake time, the next scheduled medicine intake time, and a current time.

2. The medicine intake support device according to claim 1, wherein the processing circuitry is further configured to
acquire, as properties of the prescription medicine, at least one of
a severity of side effects of the prescription medicine,
a likelihood of the side effects of the prescription medicine,
an effective blood concentration of the prescription medicine, and
an effective tissue concentration of the prescription medicine; and
suggest the response method further based on the properties of the prescription medicine.

3. The medicine intake support device according to claim 2, wherein the processing circuitry is further configured to
acquire, as the likelihood of the side effects, at least one of
a blood concentration time curve showing a change of blood concentration of the prescription medicine over time,
a blood concentration of the prescription medicine possibly causing the side effects, and
a tissue concentration of the prescription medicine possibly causing the side effects.

4. The medicine intake support device according to claim 2, wherein the processing circuitry is further configured to
acquire the properties of the prescription medicine, the properties having contents corresponding to characteristics of the patient.

5. The medicine intake support device according to claim 4, wherein the processing circuitry is further configured to
acquire, as the properties of the prescription medicine, the properties having contents corresponding to at least one of
an age of the patient,
a constitution of the patient, and
a medicine which the patient takes as the prescription medicine.

6. The medicine intake support device according to claim 1, wherein the processing circuitry is further configured to
detect that the patient took the prescription medicine; and
acquire, as the previous medicine intake time, a time of detecting that the patient took the prescription medicine.

7. The medicine intake support device according to claim 2, wherein the processing circuitry is further configured to
calculate a next recommended medicine intake time based on the properties of the prescription medicine, the previous medicine intake time, the next scheduled medicine intake time, and the current time, wherein the next recommended medicine intake time is a time recommended for the patient to take the next dose of the prescription medicine; and
suggest the response method based on the next recommended medicine intake time that was calculated.

8. The medicine intake support device according to claim 2, wherein the processing circuitry is further configured to suggest the response method to an information terminal device of at least one of a healthcare professional and the patient.

9. The medicine intake support device according to claim 7, wherein the processing circuitry is further configured to
acquire a previous scheduled medicine intake time, wherein the previous scheduled medicine intake time is a time the patient was scheduled to take the prescription medicine;
detect that the patient failed to take the prescription medicine on the previous scheduled medicine intake time based on the previous medicine intake time and the previous scheduled medicine intake time; and
suggest the response method when detecting that the patient failed to take the prescription medicine.

10. The medicine intake support device according to claim 9, wherein the processing circuitry is further configured to
suggest as the response method, if the current time is calculated as the next recommended medicine intake time, to take the prescription medicine, which was failed to be taken at the previous scheduled medicine intake time, now; and
suggest as the response method, if the next scheduled medicine intake time is calculated as the next recommended medicine intake time, to take the prescription medicine, which was failed to be taken at the previous scheduled medicine intake time, on the next scheduled medicine intake time without taking it now.

11. The medicine intake support device according to claim 10, wherein the processing circuitry is further configured to
when detecting that the patient has failed to take the prescription medicine, encourage the patient to perform a confirmation operation of failing intake of medicine by issuing an alarm to an information terminal device of the patient, the alarm notifying the patient of failing intake of medicine;
when detecting the confirmation operation of failing intake of medicine, estimate the blood concentration of the prescription medicine at a time of detecting the confirmation operation of failing intake of medicine and a maximum blood concentration of the prescription medicine when taking the prescription medicine on both the current time and the next scheduled medicine intake time based on the properties of the prescription medicine; and
determine whether to set either the current time or the next scheduled medicine intake time as the next recommended medicine intake time based on the blood concentration and the maximum blood concentration that were estimated.

12. The medicine intake support device according to claim 9, wherein the memory further stores a type of the prescription medicine and a second measure in response to excessive intake of the prescription medicine in correspondence with each other, wherein
the processing circuitry is further configured to
detect that the patient took the prescription medicine excessively based on the previous medicine intake time and the previous scheduled medicine intake time; and
if the patient took the prescription medicine excessively, suggest a response method in response to excessive intake of the prescription medicine based on the second measure stored in the memory, the previous medicine intake time, the next scheduled medicine intake time, and the current time.

13. The medicine intake support device according to claim 12, wherein the processing circuitry is further configured to
acquire at least one past medicine intake time and at least one past scheduled medicine intake time, wherein the past medicine intake time includes at least the previous medicine intake time and is a time when the patient took the prescription medicine in the past, and the past scheduled medicine intake time includes at least the previous scheduled medicine intake time and is a time when the patient was scheduled to take the prescription medicine in the past; and
detect that the patient took the prescription medicine excessively based on the at least one past medicine intake time and the at least one past scheduled medicine intake time.

14. The medicine intake support device according to claim 13, wherein the processing circuitry is further configured to
if a time after the next scheduled medicine intake time is calculated as the next recommended medicine intake time, suggest taking the prescription medicine on the time after the next scheduled medicine intake time without taking the prescription medicine on the next scheduled medicine intake time as the response method in response to excessive intake of the prescription medicine.

15. The medicine intake support device according to claim 13, wherein the processing circuitry is further configured to
when detecting that the patient has taken the prescription medicine excessively, make an inquiry of a healthcare professional about urgent medicine intake guidance with respect to the patient's excessive intake of the prescription medicine in response to the properties of the prescription medicine; and
suggest the patient the urgent medicine intake guidance responded by the healthcare professional.

16. The medicine intake support device according to claim 3, wherein the properties of the prescription medicine include properties that continues to be reacquired in response to an examination of the patient.

17. The medicine intake support device according to claim 6, wherein the processing circuitry is further configured to
detect that the prescription medicine was taken in response to an electric signal generated from a sensor attached to the prescription medicine itself when the prescription medicine was taken.

18. The medicine intake support device according to claim 6, wherein the processing circuitry is further configured to
detect that the prescription medicine was taken in response to a notification of completing intake of medicine from an information terminal device of the patient.

19. The medicine intake support device according to claim 11, wherein the processing circuitry is further configured to
determine whether to set either the current time or the next scheduled medicine intake time as the next recommended medicine intake time, even when the confirmation operation of failing intake of medicine voluntarily performed by the patient was detected before notifying the alarm of failing intake of medicine.

20. The medicine intake support device according to claim 15, wherein the processing circuitry is further configured to
suggest the medicine intake guidance to an information terminal of the patient.
